(19) United States
(12) Patent Application Publication
(10) Pub. No.: US 2026/0053608 A1
(43) Pub. Date: Feb. 26, 2026

(54) ORAL CAVITY CLEANING APPARATUS

(71) Applicant: LG H&H CO., LTD., Seoul (KR)

(72) Inventor: Jeongeun Heo, Seoul (KR)

(73) Assignee: LG H&H CO., LTD., Seoul (KR)

(21) Appl. No.: 19/306,706

(22) Filed: Aug. 21, 2025

(30) Foreign Application Priority Data
Aug. 22, 2024 (KR) 10-2024-0113084

(51) Int. Cl. A61C 17/024; A61C 17/22; A61N 5/06

(52) U.S. Cl. CPC A61C 17/024; A61C 17/22; A61N 5/0603; A61N 2005/0606; A61N 2005/0651

(57) ABSTRACT

An oral cavity cleaning apparatus according to an embodiment of the present disclosure comprises a body portion accommodating a driving portion therein, a nozzle portion disposed at one end of the body portion and comprising a through hole, and a cover member covering the through hole in the nozzle portion.

### CROSS-REFERENCE TO RELATED APPLICATION

The present application claims priority under 35 U.S.C. § 119 (a) to Korean patent application number 10-2024-0113084 filed on Aug. 22, 2024 in the Korean Intellectual Property Office, the entire disclosure of which is incorporated by reference herein.

### BACKGROUND

### 1. Technical Field

The present disclosure relates to an oral cavity cleaning apparatus.

### 2. Related Art

An oral cavity cleaning apparatus can be used to maintain oral hygiene and remove foreign substances from the user's mouth. However, when the oral cavity cleaning apparatus is used in public places, saliva may spread to surrounding people, which may be considered unhygienic behavior.

In addition, when attempting to maintain oral cleanliness while traveling or before a meeting, it is often difficult to find a restroom or there may be insufficient time.

Furthermore, using existing oral cleaning apparatuses may also increase the risk of tooth wear.

### SUMMARY

An aspect of the present disclosure is to provide an oral cleaning apparatus that allows for quick and convenient oral cleansing.

In addition, another aspect of the present disclosure is to provide an oral cleaning apparatus that comprises not only cleansing but also teeth whitening, antibacterial, and disinfection functions.

In addition, aspect of the present disclosure is to provide an oral cleaning apparatus that is easy to carry and is stored hygienically.

According to an embodiment, an oral cavity cleaning apparatus may comprise a body portion accommodating a driving portion therein, a nozzle portion disposed at one end of the body portion and comprising a through hole, and a cover member covering the through hole in the nozzle portion.

The cover member may comprise a cleaning member, and a sheet member impregnated with the cleaning member.

The nozzle portion may be detachably coupled to the body portion.

The nozzle portion may comprise a groove portion on one side thereof, and the cover member is inserted into the nozzle portion through the groove portion.

The nozzle portion may comprise a first leg portion and a second leg portion connected to the body portion, and the first leg portion and the second leg portion may be disposed to face each other and may be movable in a direction toward or away from each other.

The cover member may be disposed between the first leg portion and the second leg portion.

The nozzle portion may be rotatably coupled to the body portion.

According to an embodiment, an oral cavity cleaning apparatus may comprise a body portion accommodating a driving portion therein, a cartridge portion disposed in a receiving space provided in the body portion and accommodating a cleaning member in liquid form, and a discharge portion connected to the cartridge portion and discharging the cleaning member.

The discharge portion may comprise: a discharge cover portion forming an outer shape of the discharge portion and allowing the cleaning member to move inward, and a discharge body portion in communication with an inside of the discharge cover portion and comprising a discharge hole through which the cleaning member is sprayed outward.

The oral cleaning apparatus may further comprise a mounting portion provided by surrounding at least a portion of an outer surface by the body portion, wherein the discharge portion is disposed at the mounting portion.

The discharge portion may be slidably coupled to the body portion.

The discharge portion may be hinge-connected to the body portion.

The body portion may further comprise a body cover portion opening or closing the receiving portion.

The oral cleaning apparatus may further comprise an irradiation portion irradiating light of a predetermined wavelength.

Each of the discharge portion and the irradiation portion may be disposed at one end of the body portion.

### BRIEF DESCRIPTION OF THE DRAWINGS

FIG. 1 illustrates an oral cleaning apparatus according to an embodiment of the present disclosure;

FIG. 2 illustrates a nozzle portion according to an embodiment of the present disclosure;

FIGS. 3 and 4 illustrate the interior of an oral cleaning apparatus according to an embodiment of the present disclosure;

FIGS. 5 to 7 illustrate a nozzle portion according to various embodiments of the present disclosure;

FIG. 8 illustrates an oral cleaning apparatus according to another embodiment of the present disclosure;

FIG. 9 illustrates an oral cleaning apparatus according to another embodiment of the present disclosure;

FIGS. 10 and 11 are exploded views of an oral cleaning apparatus according to another embodiment of the present disclosure;

FIGS. 12 and 13 are exploded views of a discharge portion of an oral cleaning apparatus according to another embodiment of the present disclosure;

FIG. 14 illustrates an oral cleaning apparatus according to another embodiment of the present disclosure;

FIGS. 15A to 15J illustrate an oral cleaning apparatus according to various embodiments of the present disclosure;

FIG. 16 illustrates an oral cleaning apparatus according to another embodiment of the present disclosure;

FIG. 17 illustrates an oral cleaning apparatus according to another embodiment of the present disclosure;

FIG. 18 illustrates an oral cleaning apparatus according to another embodiment of the present disclosure; and

FIGS. 19A and 19B illustrate an example of a state in which an oral cleaning apparatus is used.

### DETAILED DESCRIPTION

Hereinafter, the present disclosure will be described in detail with reference to the attached drawings. This is merely illustrative, and the present disclosure is not limited to the specific embodiments described in an illustrative manner.

Specific terms used in the present specification are merely for convenience of explanation and are not used to limit the illustrated embodiments.

For example, expressions such as “same” and “is the same” not only indicate a strictly identical state, but also indicate a state in which there is a difference in tolerance or the degree to which the same function is obtained.

For example, expressions representing relative or absolute arrangement such as “in a certain direction,” “along a certain direction,” “side by side,” “perpendicularly,” “at the center,” “concentric,” or “coaxial,” not only strictly represent the arrangement, but also represent the state of relative displacement with a tolerance, or an angle or distance at which the same function is obtained.

To explain the present disclosure, descriptions below may be based on a spatial orthogonal coordinate system with X, Y, and Z axes orthogonal to each other. Each axis direction (X-axis direction, Y-axis direction, Z-axis direction) refers to both directions in which each axis extends.

The X-direction, Y-direction, and Z-direction mentioned below are for the purpose of explanation, so that the present disclosure may be clearly understood. The directions may be defined differently depending on where the reference is placed.

The use of terms such as ‘first, second, and third’ in front of the components mentioned below is only to avoid confusion about the components to which they are referred and is irrelevant to the order, importance, or master-slave relationship between the components, etc. For example, an embodiment that comprises only a second component without a first component may also be implemented.

The terms used in this disclosure are intended to describe specific embodiments and are not intended to limit the scope of the claims. As used in the present specification, singular expressions comprise plural expressions unless the context clearly dictates otherwise.

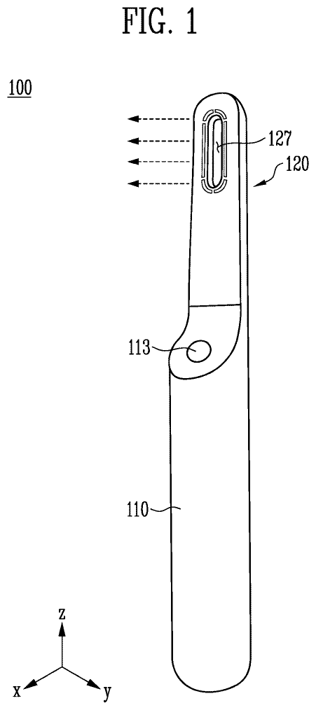

FIG. 1 illustrates an oral cleaning apparatus 100 according to an embodiment of the present disclosure.

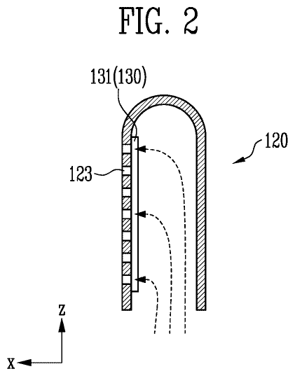

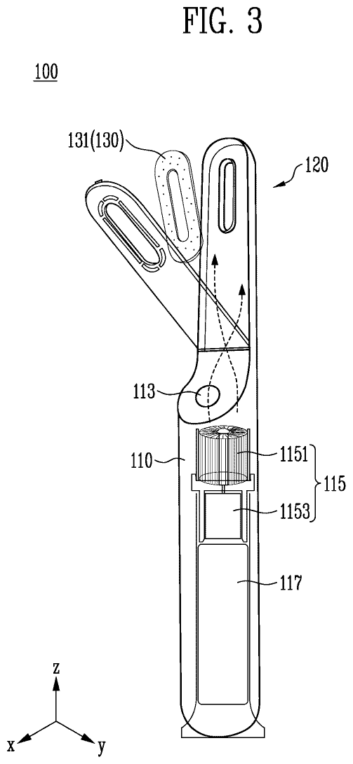

The oral cleaning apparatus 100 according to the present disclosure comprises a body portion 110 which internally accommodates a driving portion 115 as shown in FIG. 3, a nozzle portion 120 disposed at one end of the body portion 110 and comprising a through hole 123 as shown in FIG. 2, and a cover member 130 covering the through hole 123 within the nozzle portion 120 as shown in FIG. 2.

The body portion 110 may form the exterior of the oral cleaning apparatus 100. The body portion 110 may have a portable configuration, which means that the body portion 110 may have a size, weight, etc., which generally meets the criteria for portability.

The body portion 110 may have various shapes to allow a user to grip the body portion 110. For example, the body portion 110 may have a cylindrical shape. The body portion 110 may have a curved surface to improve portability.

Referring to FIG. 1, the nozzle portion 120 may be positioned at one end of the body portion 110. The nozzle portion 120 may be positioned to face the user's mouth or teeth. The user may position the nozzle portion 120 to face the mouth while holding the body portion 110. In this manner, a cleaning member 132 discharged from the nozzle portion 120 may clean the user's mouth or teeth.

In an embodiment, the nozzle portion 120 may have a slender shape to facilitate insertion into the mouth. The body portion 110 may be formed with a predetermined thickness to facilitate gripping by the user, and the nozzle portion 120 may be formed with a thickness smaller than that of the body portion 110.

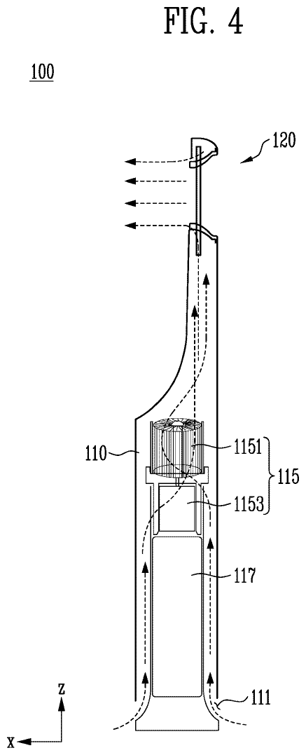

The body portion 110 may further comprise an inlet portion 111 as shown in FIG. 4. Air from the outside may enter the interior of the body portion 110 through the inlet portion 111. The air which enters the interior of the body portion 110 through the inlet portion 111 may be discharged to the outside of the body portion 110 through the nozzle portion 120.

In an embodiment, the inlet portion 111 may be provided on the opposite side of the nozzle portion 120. The inlet portion 111 is formed at one end of the body portion 110, and the nozzle portion 120 may be formed at the other end of the body portion 110. In this case, air will move from the inlet portion 111 toward the nozzle portion 120.

To increase the air pressure, the thicknesses of the body portion 110 and the nozzle portion 120 may be formed differently. The thickness of the body portion 110 may differ from that of the nozzle portion 120. The thickness of the body portion 110 may be greater than the thickness of the nozzle portion 120. As the air moves toward the nozzle portion 120, an airflow path narrows, thereby increasing the airflow velocity.

Referring to FIG. 1, the nozzle portion 120 may comprise a nozzle hole 127. The nozzle hole 127 may be formed to pass through the nozzle portion 120. Due to the nozzle hole 127, the air inside the body portion 110 may be divided into two streams and move, thereby increasing the air velocity.

The cleaning member 132 may be discharged from one surface of the nozzle portion 120. Therefore, the user may position the nozzle portion 120 such that one surface thereof may face the oral cavity, and may then press a button portion 113 to discharge the cleaning member 132 into the oral cavity.

The oral cleaning apparatus 100 may further comprise the button portion 113. The oral cleaning apparatus 100 may be powered on or off by the button portion 113. The button portion 113 may be formed on one surface of the body portion 110. The button portion 113 may be formed at a position where it is easy for the user to grip and press the body portion 110 at the same time. Referring to FIG. 1, the button portion 113 may be formed at a position where the thumb touches the button portion 113 when the user grips the body portion 110 with four fingers excluding the thumb.

In an embodiment, pressing the button portion 113 may initiate the operation of the oral cleaning apparatus 100. In another embodiment, the button portion 113 may comprise a touch sensor to operate in response to the user's touch.

In an embodiment, the nozzle portion 120 may be provided as a separate structure from the body portion 110. The nozzle portion 120 may be configured to be detachably coupled to the body portion 110.

In another embodiment, the nozzle portion 120 may be integrally formed with the body portion 110. In this case, the nozzle portion 120 may be positioned at one end of the body portion 110.

FIG. 2 illustrates the nozzle portion 120 according to an embodiment of the present disclosure.

The through hole 123 may be formed on one surface of the nozzle portion 120. Air may move between the interior and exterior of the nozzle portion 120 through the through hole 123. The through hole 123 may be provided as a plurality of through holes. One surface of the nozzle portion 120 with the through hole 123 may be positioned to face the user's oral cavity.

The oral cleaning apparatus 100 may spray the cleaning member 132 outward along with air. The cleaning member 132 may clean, disinfect, and sterilize the mouth. In addition, the cleaning member 132 may comprise an ingredient having at least one function selected from fragrance emission, whitening, antibacterial activity, and disinfection.

To spray the cleaning member 132, the oral cleaning apparatus 100 according to the present disclosure comprises the cover member 130. The cover member 130 may cover the through hole 123 inside the nozzle portion 120. Since the cover member 130 covers the through hole 123, air may pass through the cover member 130 and be discharged outside the nozzle portion 120 via the through hole 123.

The cover member 130 may comprise the cleaning member 132 and a sheet member 131 impregnated with the cleaning member 132. The sheet member 131 may be impregnated with the cleaning member 132. The sheet member 131 may be formed from a material capable of impregnating the cleaning member 132. For example, the sheet member 131 may be a non-woven fabric.

The sheet member 131 may impregnate the cleaning member 132, and the cleaning member 132 may be sprayed in all directions by air. In other words, when air at a predetermined pressure is applied to the sheet member 131, the cleaning member 132 impregnated in the sheet member 131 may be separated from the sheet member 131 and sprayed out.

Referring to FIG. 2, the sheet member 131 may cover the through hole 123. In addition, the cleaning member 132 impregnated in the sheet member 131 may be discharged outside the through hole 123 at the same time as air passes through the sheet member 131 and is discharged outside the through hole 123, whereby the cleaning member 132 may be sprayed into the user's mouth.

The cleaning member 132 may be sprayed in an aerosol form. The cleaning member 132 in the aerosol form may be effectively sprayed within the oral cavity. In addition, since oxygen is compressed to form the aerosol, the cleaning member 132 may be discharged with strong pressure.

Meanwhile, as shown in FIG. 2, the sheet member 131 is attached to one surface of the nozzle portion 120, but the sheet member 131 may be positioned separately from the nozzle portion 120. The sheet member 131 may only be positioned in front of the through hole 123 along an air discharge path.

FIGS. 3 and 4 illustrate the internal structure of the oral cleaning apparatus 100 according to an embodiment of the present disclosure. Specifically, FIGS. 3 and 4 show the internal components of the oral cleaning apparatus 100 for convenience of explanation.

The body portion 110 may provide an internal space capable of accommodating various configurations for the operation of the oral cleaning apparatus 100. The body portion 110 may accommodate the driving portion 115 internally.

The driving portion 115 may provide power to discharge air outward. The driving portion 115 may generate airflow within the body portion 110 and spray air with strong force.

In an embodiment, the driving portion 115 may comprise a motor 1153 and a blade portion 1151. When the button portion 113 is pressed by the user, the motor 1153 and the blade portion 1151 may operate. The motor 1153 may provide power for the rotation of the blade portion 1151, and air may be discharged from the interior of the body portion 110 toward the exterior by the blade portion 1151.

In addition, the oral cleaning apparatus 100 may further comprise a control portion 117. The control portion 117 may comprise a substrate portion and control the operation of the oral cleaning apparatus 100. For example, when the button portion 113 is pressed, the substrate portion may receive a press signal and the driving portion 115 may be operated through the press signal.

The oral cleaning apparatus 100 may further comprise a battery portion (not shown). The battery portion may be a rechargeable battery capable of repeated charging and discharging. In this case, the oral cleaning apparatus 100 may be provided with a terminal portion (not shown) on one side thereof. The battery portion may be charged by an external power supply apparatus through a terminal portion.

In another embodiment, the battery portion may be a replaceable battery. After the battery portion has been used to a certain extent, the battery portion may be replaced with a new battery portion.

Referring to FIGS. 3 and 4, when the driving portion 115 operates, air may be drawn into the inlet portion 111 at one end of the body portion 110. The air drawn into the interior of the body portion 110 may move toward the nozzle portion 120. The air flowing toward the nozzle portion 120 may accelerate in speed due to the narrowed path and may be discharged outward through the sheet member 131. In addition, the cleaning member impregnated in the sheet member 131 may be discharged outward along with the air.

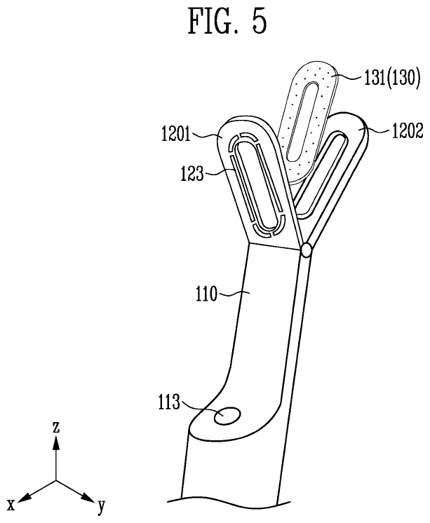

FIGS. 5 to 7 illustrate the nozzle portion 120 according to various embodiments of the present disclosure. FIGS. 5 to 7 show oral cleaning apparatus 100 which is the same as the aforementioned oral cleaning apparatus 100 except for the nozzle portion 120, so only the configuration of the nozzle portion 120 will be described.

In an embodiment, the nozzle portion 120 may comprise a first leg portion 1201 and a second leg portion 1202 connected to the body portion 110. The first leg portion 1201 and the second leg portion 1202 may be arranged to face each other and may be movable in a direction toward or away from each other.

Referring to FIG. 5, one end of the first leg portion 1201 may be connected to the body portion 110. The first leg portion 1201 may rotate around one end of the first leg portion 1201.

In addition, one end of the second leg portion 1202 may be connected to the body portion 110. The second leg portion 1202 may rotate around the center of one end of the second leg portion 1202.

The first leg portion 1201 and the second leg portion 1202 may be disposed facing each other and may be movable in a direction toward or away from each other.

The cover member 130 may be disposed between the first leg portion 1201 and the second leg portion 1202. The cover member 130 may be disposed between the first leg portion 1201 and the second leg portion 1202 while the first leg portion 1201 and the second leg portion 1202 are moved apart from each other. After the cover member 130 is placed, the first leg portion 1201 and the second leg portion 1202 may be moved closer to each other. The first leg portion 1201 and the second leg portion 1202 may be connected to each other along corners thereof. At this time, the first leg portion 1201 and the second leg portion 1202 may be airtightly connected.

The through hole 123 may be formed in at least one of the first leg portion 1201 and the second leg portion 1202. Referring again to FIG. 5, the through hole 123 may be formed in the first leg portion 1201, and air may be discharged to the outside through the through hole 123 of the first leg portion 1201.

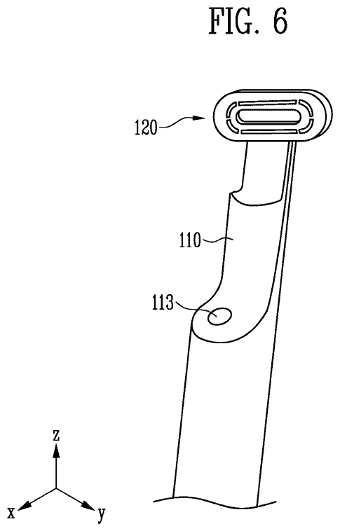

In another embodiment, the nozzle portion 120 may be rotatably coupled to the body portion 110. The nozzle portion 120 may be rotatably coupled to one end of the body portion 110. The nozzle portion 120 may rotate about an axis directed toward the oral cavity. Referring to FIG. 6, the nozzle portion 120 may be disposed toward the oral cavity along an X-axis direction. At this time, the nozzle portion 120 may be rotatable about the X-axis direction. The rotation of the nozzle portion 120 may allow the nozzle portion 120 to be easily inserted into the oral cavity or efficiently clean the oral cavity.

In another embodiment, the nozzle portion 120 comprises a groove portion 125 on one side thereof, and the cover member 130 may be inserted into the nozzle portion 120 through the groove portion 125. The groove portion 125 may be formed in a slit shape. The groove portion 125 may be formed to a size sufficient for the cover member 130 to be inserted.

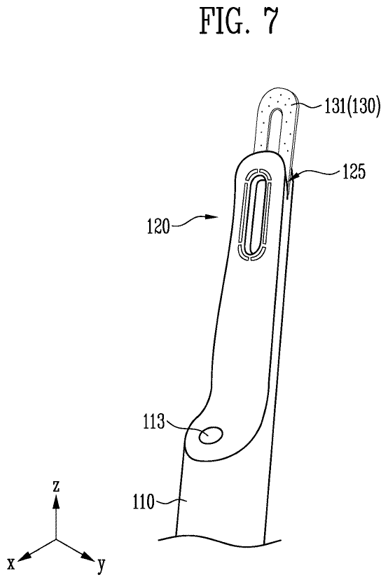

Referring to FIG. 7, the groove portion 125 is formed at one end of the nozzle portion 120, and the cover member 130 may be inserted through the groove portion 125. Meanwhile, the oral cleaning apparatus 100 may further comprise a cap portion (not shown) for closing the groove portion 125 after the cover member 130 is inserted into the groove portion 125. By closing the groove portion 125 with the cap portion, air may be efficiently discharged through the through hole 123.

When the cleaning member 132 impregnated in the cover member 130 is depleted, the cover member 130 inserted into the groove portion 125 may be removed from the oral cleaning apparatus 100 after the cap portion is opened. The oral cleaning apparatus 100 may be used by reinserting a new cover member 130 into the groove portion 125 and closing the cap portion.

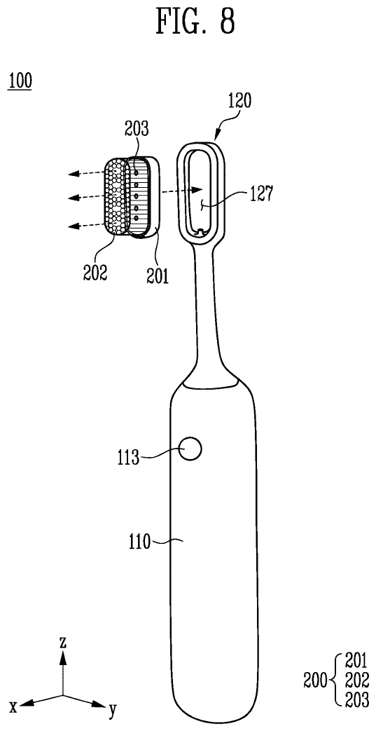

FIG. 8 illustrates the oral cleaning apparatus 100 according to another embodiment of the present disclosure.

The oral cleaning apparatus 100 may further comprise a brush portion 200. The brush portion 200 may come into contact with teeth to physically clean the teeth. The brush portion 200 may comprise a brush body 201 and brush bristles 202. The brush body 201 may form the outer shape of the brush portion 200. The brush bristles 202 are attached to one surface of the brush portion 200 and are capable of cleaning the oral cavity. The brush bristles 202 may have a general degree of elasticity and roughness to remove foreign substances without damaging the teeth.

The brush portion 200 may be coupled to the nozzle portion 120. The brush portion 200 may be coupled to the nozzle hole 127. Referring to FIG. 8, the edge of the nozzle hole 127 may be formed adjacent to the edge of the nozzle portion 120, and the nozzle portion 120 may form a thin ring shape. The brush body 201 may be coupled to the inside of the ring of the nozzle portion 120.

The brush body 201 may comprise a space through which air flows. The interior of the brush body 201 may be in communication with the interior of the nozzle portion 120, whereby air inside the body portion 110 may move into the interior of the brush portion 200. The brush body 201 may comprise a brush hole 203. The brush hole 203 may be formed on one surface of the brush body 201. The brush hole 203 may be formed on the same surface as the surface where the brush bristles 202 are formed.

Ultimately, when the brush portion 200 is coupled, the brush bristles 202 clean the oral cavity while air is discharged outward through the brush hole 203, thereby enhancing the cleaning effect.

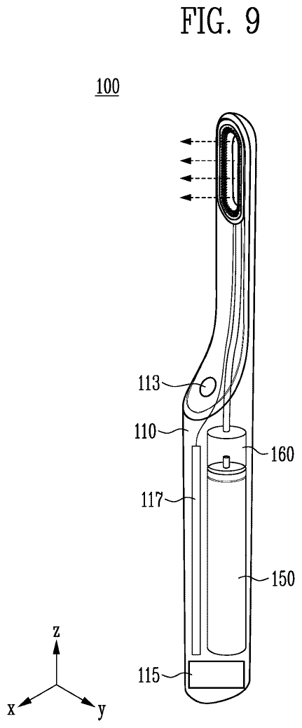

FIG. 9 illustrates the oral cleaning apparatus 100 according to another embodiment of the present disclosure.

The oral cleaning apparatus 100 according to another embodiment of the present disclosure comprises the body portion 110 which accommodates the driving portion 115 internally, a cartridge portion 150 positioned in a receiving space formed within the body portion 110 and accommodating the cleaning member 132, and a discharge portion 160 connected to the cartridge portion 150 and discharging the cleaning member 132.

Specifically, for the convenience of explanation, FIG. 9 shows the internal components of the oral cleaning apparatus 100. The oral cleaning apparatus 100 according to another embodiment of the present disclosure differs from the aforementioned oral cleaning apparatus 100 in which the oral cleaning apparatus 100 comprises the cartridge portion 150 and the discharge portion 160.

The cartridge portion 150 may accommodate the cleaning member 132 in liquid form. The liquid cleaning member 132 accommodated in the cartridge portion 150 may be discharged outward through the discharge portion 160. The discharge portion 160 may be connected to the cartridge portion 150. The discharge portion 160 may be positioned to face the user's oral cavity. The cleaning member 132 discharged from the discharge portion 160 may clean the user's oral cavity.

In another embodiment of the present disclosure, the driving portion 115 may discharge the cleaning member 132 received in the cartridge portion 150 to the outside. That is, the driving portion 115 may provide the power to move the cleaning member inside the cartridge portion to the outside, rather than generating an air flow.

In an embodiment, the cartridge portion 150 may be an aerosol spray. In this case, the driving portion 115 may be integrally formed with the cartridge portion 150. When the user presses the button portion, the cleaning member received in the cartridge portion 150 may move outward from the cartridge portion by the driving portion 115.

Referring to FIG. 9, the cartridge portion 150 and the discharge portion 160 may be positioned inside the body portion 110. In an embodiment, when the user presses the button portion 113, the cleaning member 132 received in the cartridge portion 150 may be discharged through the discharge portion 160.

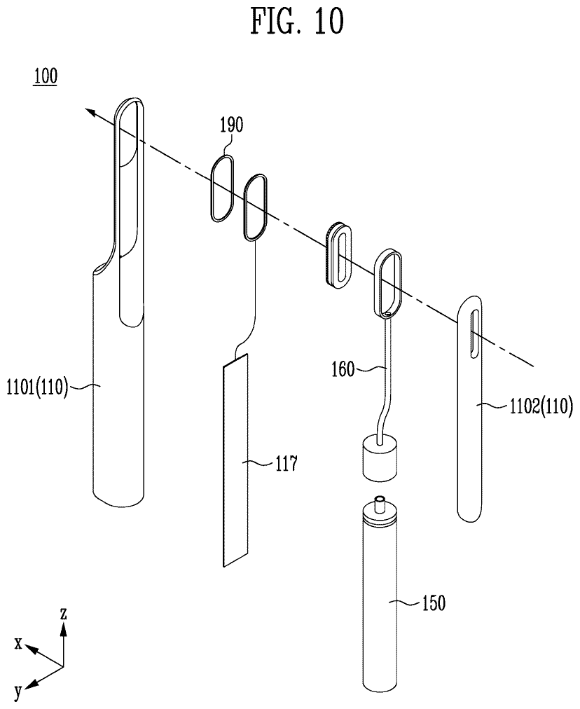

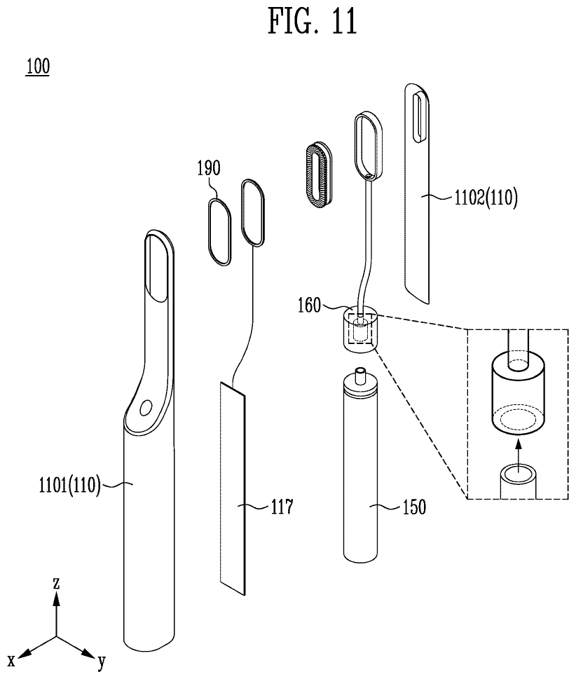

FIGS. 10 and 11 are exploded views of the oral cleaning apparatus 100 according to another embodiment of the present disclosure.

The body portion 110 may comprise a first body portion 1101 and a second body portion 1102. The first body portion 1101 and the second body portion 1102 may be connected to each other. The oral cleaning apparatus 100 may be assembled by combining the second body portion 1102 with the first body portion 1101 after each component is inserted into the first body portion 1101.

Referring to FIGS. 10 and 11, the first body portion 1101 is provided with a rear surface thereof opened, and the cartridge portion 150 and the discharge portion 160 may be inserted into the interior of the first body portion 1101 through the open surface. Thereafter, the second body portion 1102 may close the open rear surface of the first body portion 1101.

In addition, the oral cleaning apparatus 100 may further comprise an irradiation portion 190. The irradiation portion 190 may emit light at a predetermined wavelength. In an embodiment, the irradiation portion 190 is capable of emitting LED light. The oral cleaning apparatus of the present disclosure may impart antibacterial and whitening effects to the oral cavity through the LED lighting.

Each of the discharge portion 160 and the irradiation portion 190 may be disposed at one end of the body portion 110. The irradiation portion 190 may be formed adjacent to the discharge portion 160. When the discharge portion 160 is positioned toward the oral cavity, the irradiation portion 190 may also be positioned toward the oral cavity. When the cleaning member 132 is ejected from the discharge portion 160, the irradiation portion 190 may emit light of a predetermined wavelength toward the oral cavity, thereby enhancing the cleaning, antibacterial, and whitening effects.

Referring to FIG. 11, the body portion 110 may further comprise the control portion 117. The control portion 117 comprises a substrate portion which receives operation signals input from the button portion 113. In addition, the substrate portion may be connected to the irradiation portion 190, and the control portion 117 may control the irradiation portion 190.

The cartridge portion 150 and the discharge portion 160 may be connected. In an embodiment, the cartridge portion 150 and the discharge portion 160 may be connected in a one-touch structure. More specifically, the cartridge portion 150 may comprise an engagement portion (not shown). When the discharge portion 160 is inserted into the cartridge portion 150, the engagement portion facilitates easy coupling between the cartridge portion 150 and the discharge portion 160. Conversely, the discharge portion 160 may also comprise an engagement member.

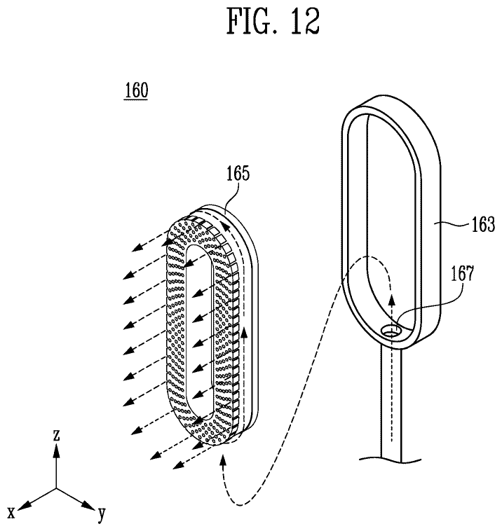

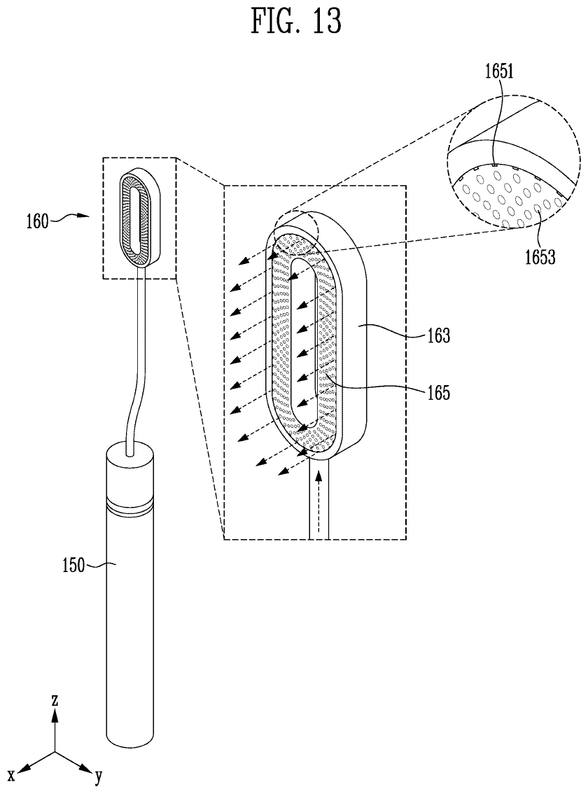

FIGS. 12 and 13 are exploded views of the discharge portion 160 of the oral cleaning apparatus 100 according to another embodiment of the present disclosure.

The discharge portion 160 may comprise a discharge cover portion 163 and a discharge body portion 165. The discharge cover portion 163 forms the outer shape of the discharge portion 160 and allows the cleaning member 132 to move inward.

The discharge body portion 165 may communicate with the interior of the discharge cover portion 163 and may comprise a discharge hole 1651 through which the cleaning member 132 is discharged outward. Ultimately, the cleaning member 132 housed in the cartridge body 150 may move through the discharge cover body 163 and the discharge body 165 and may be discharged to the outside through the discharge hole 1651.

Referring to FIG. 12, the discharge cover portion 163 may comprise a connection hole 167 on an inner surface thereof, and the connection hole 167 may connect the interior of the discharge body portion 165 to the interior of the discharge cover portion 163. At this time, the discharge cover portion 163 and the discharge body portion 165 may be tightly coupled to prevent the cleaning member 132 from leaking through a gap between the discharge cover portion 163 and the discharge body portion 165. The discharge body portion 165 may form a passageway through which the cleaning member 132 passes.

Referring to FIG. 13, the discharge hole 1651 may be formed on one surface of the discharge body portion 165. The discharge hole 1651 may be provided as a plurality of discharge holes. For example, the discharge holes 1651 may be provided in tens to hundreds, and the cleaning member 132 may be efficiently discharged through the plurality of discharge holes 1651.

Meanwhile, the discharge body portion 165 may further comprise a decorative hole 1653. The decorative hole 1653 is formed in a shape similar to that of the discharge hole 1651. However, the decorative hole 1653 does not discharge the cleaning member 132 to the outside. In an embodiment, the discharge hole 1651 may be formed along the outer side of the discharge body portion 165, and the decorative hole 1653 may be formed inside the discharge hole 1651.

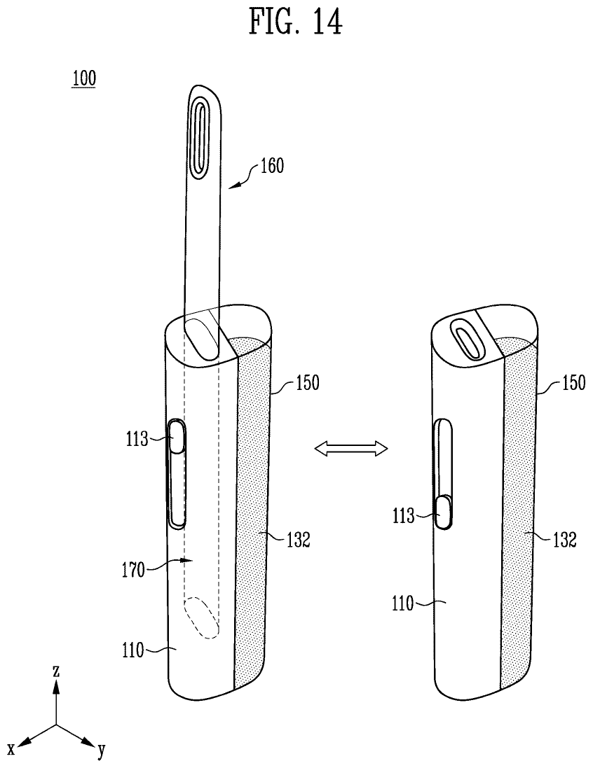

FIG. 14 illustrates the oral cleaning apparatus 100 according to another embodiment of the present disclosure.

According to another embodiment of the present disclosure, the oral cleaning apparatus 100 may further comprise a mounting portion 170. The mounting portion 170 may be formed such that at least a part of an outer surface thereof may be surrounded by the body portion 110. In other words, the mounting portion 170 may be formed by the outer surface of the body portion 110.

The discharge portion 160 may be positioned on the mounting portion 170. The discharge portion 160 may be movably provided. In an embodiment, the discharge portion 160 may be slidably coupled to the body portion 110. Referring to FIG. 14, the position of the discharge portion 160 may be moved by the button portion 113. When the button portion 113 is moved in a first direction (e.g., an upward direction), the discharge portion 160 may move to a first position. When the button portion 113 is moved in a second direction (e.g., a downward direction), the discharge portion 160 may move to a second position.

The discharge portion 160 may move repeatedly between the first position and the second position. The mounting portion 170 may form the first position where the discharge portion 160 is disposed. When the discharge portion 160 is positioned at the mounting portion 170, the discharge portion 160 is surrounded by the body portion 110, thereby preventing the discharge portion 160 from being exposed to the outside.

When the discharge portion 160 is positioned at the second position, the discharge portion 160 may be exposed to the exterior. The user may use the oral cleaning apparatus 100 by moving the discharge portion 160 to the second position and orienting the discharge portion 160 toward the mouth. The user may store the oral cleaning apparatus 100 after moving the discharge portion 160 to the first position. Referring to FIG. 14, the cleaning member 132 disposed in the cartridge portion 150 may be discharged to the outside through the discharge portion 160.

In addition, referring to FIG. 14, the cartridge portion 150 is positioned on one side (e.g., the right side), enabling the user to visually confirm the remaining amount of the cleaning member 132.

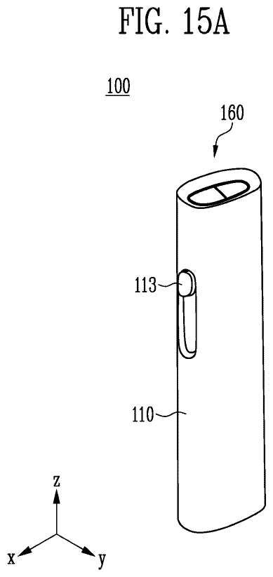

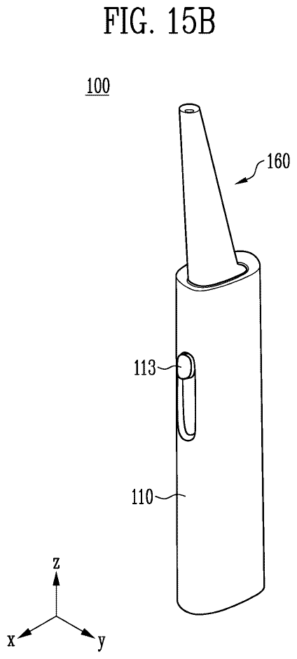

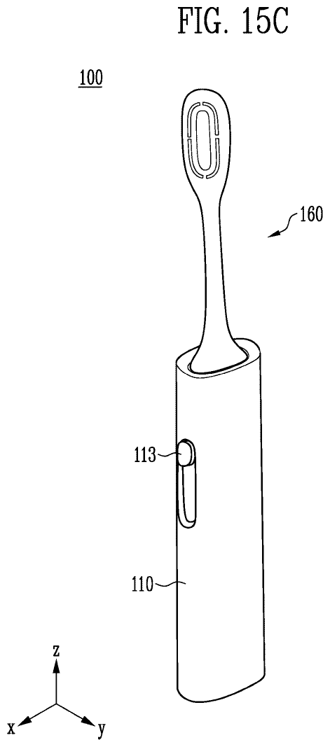

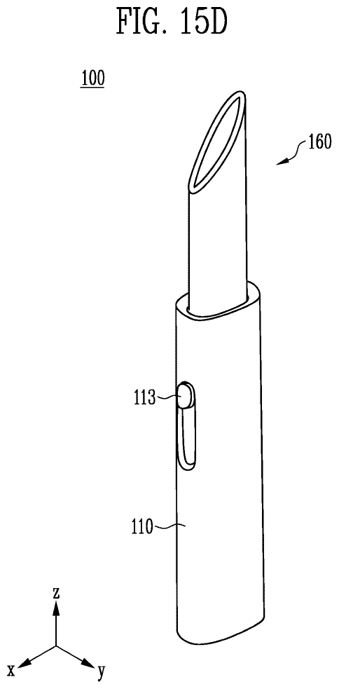

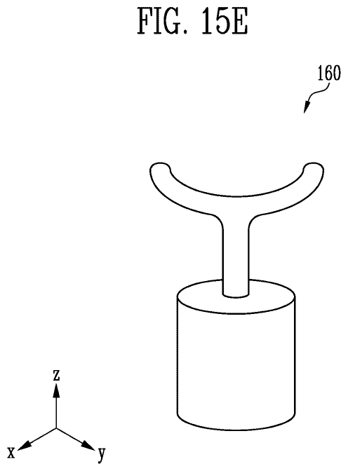

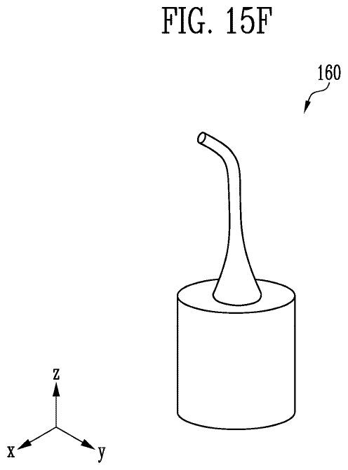

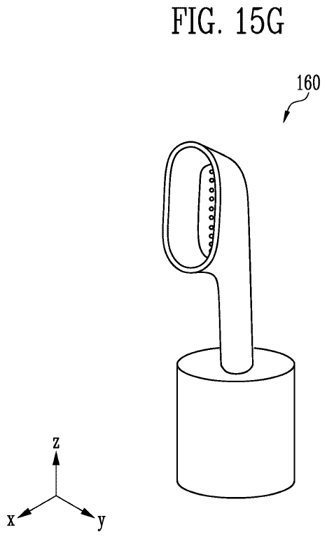

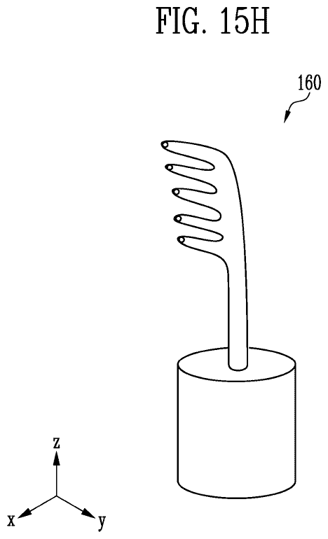

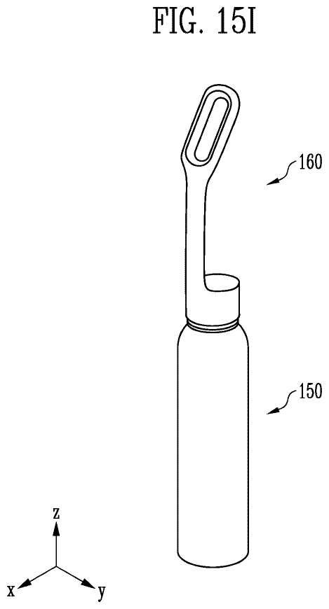

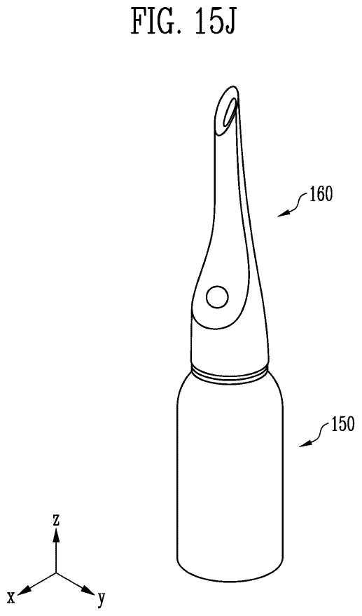

FIGS. 15A to 15J illustrate various embodiments of the oral cleaning apparatus 100.

Specifically, FIGS. 15A to 15D illustrate various shapes of the discharge portion 160. Referring to FIG. 15A, the cartridge portion 150 may be provided inside the body portion 110, and the cartridge portion 150 may be surrounded by the body portion 110 and not exposed to the outside.

In addition, the discharge portion 160 may be disposed inside the body portion 110 or may slide to be exposed outside the body portion 110. FIG. 15A illustrates the discharge portion 160 positioned inside the body portion 110. However, the discharge portion 160 may be moved to a position outside the body portion 110 via the button portion 113.

The user may conveniently carry the oral cleaning apparatus 100 with the discharge portion 160 disposed inside the body portion 110. In addition, since the discharge portion 160 is not exposed externally, the oral cleaning apparatus 100 may be stored hygienically.

Referring to FIG. 15B, the discharge portion 160 may be configured to become thinner and narrower toward the tip. The discharge portion 160 is formed to be thin and slender such that the discharge portion 160 may be easily inserted into the mouth. In addition, the discharge portion 160 may be positioned toward fine gaps between the teeth, and the cleaning member 132 may be discharged between the teeth. In other words, the user may discharge the cleaning member 132 to the desired location.

Referring to FIGS. 15C and 15D, the discharge portion 160 may be provided in a toothbrush shape. One surface of the discharge portion 160 where the discharge hole 1651 is formed may be formed with a greater width so as to spray the cleaning member 132 over a wide area.

FIGS. 15E to 15H illustrate other configurations of the discharge portion 160. Referring to FIGS. 15E to 15H, the discharge portion 160 may be formed in a denture-like shape or in a thin and fine shape. In addition, the discharge portion 160 may be formed in a comb-like shape or in a shape for spraying the cleaning member over a wide area.

Meanwhile, FIGS. 15E to 15H partially illustrate the discharge portion 160. However, a lower end of the discharge portion 160 may be variously modified and engaged with the cartridge portion 150 or the body portion 110.

FIGS. 151 and 15J illustrate other configurations of the discharge portion 160. Specifically, FIGS. 151 and 15J show the discharge portion 160 engaged with the cartridge portion 150.

Referring to FIG. 151, the discharge portion 160 may be inclined at a predetermined angle. In addition, referring to FIG. 15J, the discharge portion 160 may be formed with a shape where the width thereof gradually narrows toward an outlet disposed at one end thereof.

Ultimately, the oral cleaning apparatus 100 according to the present disclosure may be configured with the discharge portion 160 of various configurations so as to achieve various functions and effects, and the discharge portions 160 may be interchangeably used.

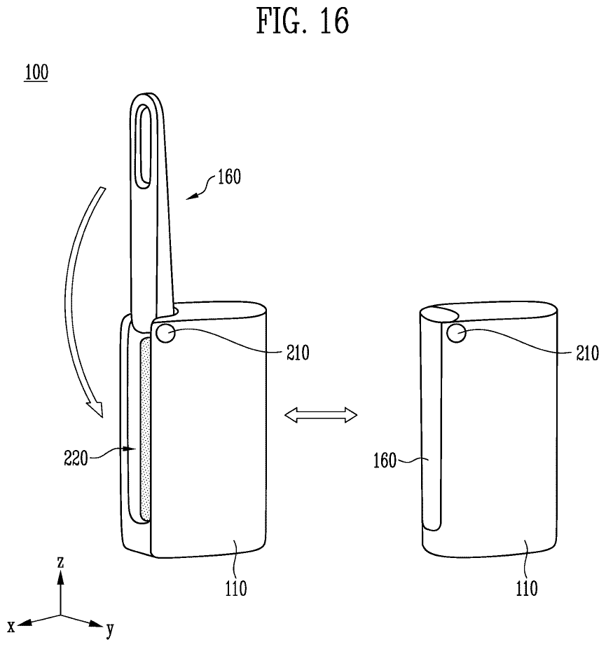

FIG. 16 illustrates the oral cleaning apparatus 100 according to another embodiment of the present disclosure.

The discharge portion 160 according to another embodiment of the present disclosure may be hinge-connected to the body portion 110. The oral cleaning apparatus 100 may further comprise a hinge portion 210. The hinge portion 210 may be engaged with one area of the body portion 110. One surface of the discharge portion 160 facing the oral cavity may rotate about the hinge portion 210 and be positioned to face the body portion 110, so that contamination of the discharge portion 160 may be prevented and the oral cleaning apparatus 100 may be conveniently carried.

Referring to FIG. 16, the body portion 110 may form a recessed portion 220 where one surface of the body portion 110 is recessed. The discharge portion 160 may be positioned in the recessed portion 220. The discharge portion 160 may not protrude from the outer surface of the recessed portion 220 when the discharge portion 160 is positioned in the recessed portion 220. By preventing the discharge portion 160 from protruding from the outer surface of the body portion 110, the user may easily grip the body portion 110.

In addition, part of the recessed portion 220 may be formed from a transparent material, allowing the interior of the body portion 110 to be visually confirmed. Thus, the user may easily check the remaining amount of the cleaning member 132 by moving the discharge portion 160.

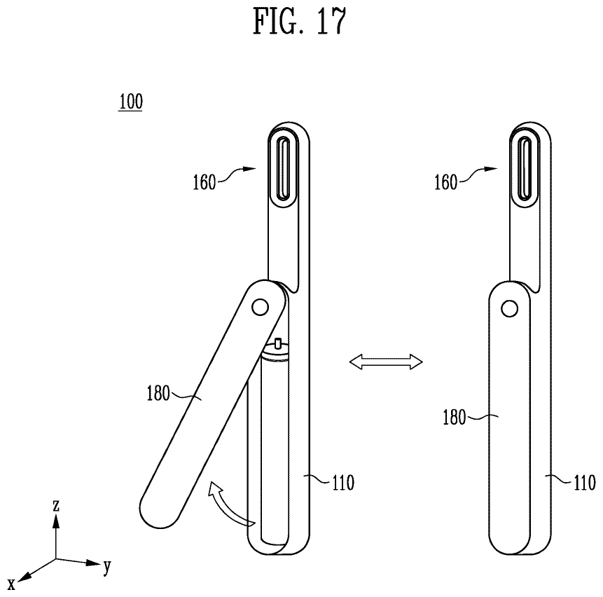

FIG. 17 illustrates another embodiment of the oral cleaning apparatus 100 according to the present disclosure.

The body portion 110 according to another embodiment of the present disclosure may comprise a body cover portion 180 which opens or closes the receiving space. The cartridge portion 150 may be positioned in the receiving space of the body portion 110.

When the body cover 180 is opened, the receiving space is opened, allowing the cartridge portion 150 to be removed. In this manner, the cartridge portion 150 may be easily replaced with a new cartridge portion 150 when the cartridge portion 150 is exhausted. When the body cover portion 180 is closed, the receiving space is closed, and the cartridge portion 150 is safely stored.

In an embodiment, the body cover portion 180 may be connected to one area of the body portion 110 via a hinge. Referring to FIG. 17, the body cover portion 180 may be hinge-connected to the central portion of the body portion 110, and the body cover portion 180 may be rotated to thereby open the receiving space. Alternatively, such hinge connection may be replaced with sliding connection or another type of connection.

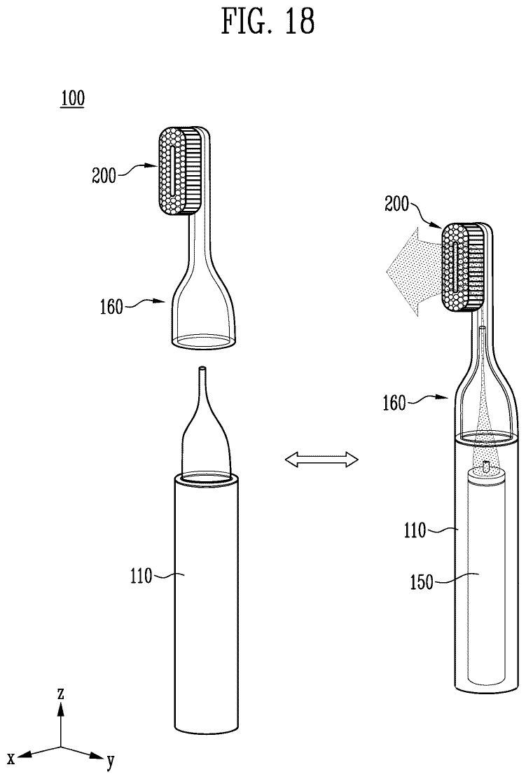

FIG. 18 illustrates the oral cleaning apparatus 100 according to another embodiment of the present disclosure.

The oral cleaning apparatus 100 according to another embodiment of the present disclosure may comprise the discharge portion 160 which is detachably coupled to the body portion 110. When the discharge portion 160 is separated from the body portion 110, the receiving space of the body portion 110 may be opened. The cartridge portion 150 may be replaced through the open surface of the body portion 110.

The discharge portion 160 may be engaged with the brush portion 200. Since the structure of the brush portion 200 is described above, further details are omitted here.

Referring to FIG. 18, the body portion 110 may be coupled to the discharge portion 160, and the discharge portion 160 may be coupled to the brush portion 200. As a result, the brush portion 200 may clean the teeth while simultaneously the cleaning member 132 is discharged, thereby enhancing cleaning, antibacterial, and whitening effects.

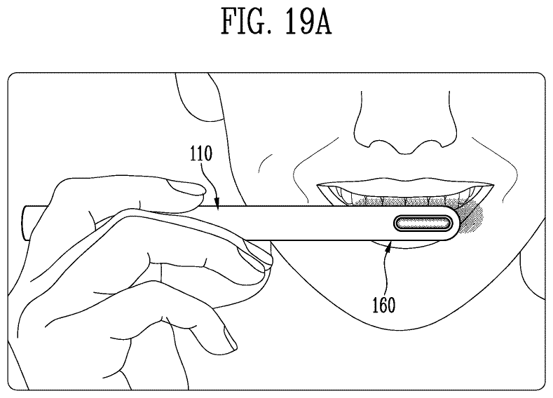

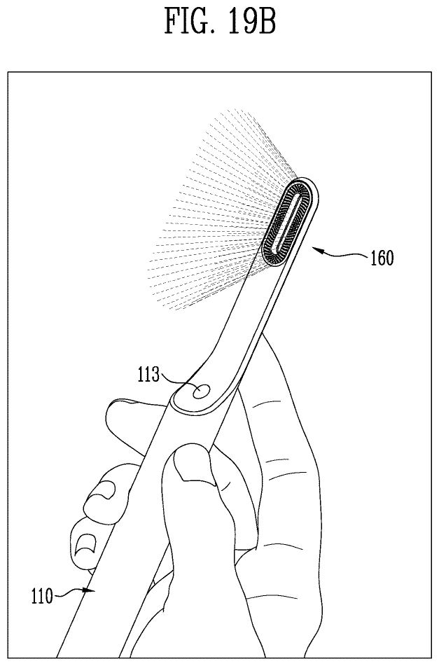

FIGS. 19A and 19B illustrate an example of the oral cleaning apparatus 100 in use.

Referring to FIGS. 19A and 19B, the user may grip the body portion 110 and position the discharge portion 160 toward the mouth. When the button portion 113 is pressed, the cleaning member 132 may be sprayed from the discharge portion 160. At the same time, LED light may be emitted from the irradiation portion 190.

According to an embodiment of the present disclosure, an oral cleaning apparatus that can clean the mouth conveniently and quickly may be provided.

In addition, the present disclosure provides an oral cleaning apparatus that comprises not only cleaning but also teeth whitening and antibacterial functions.

In addition, the present disclosure provides an oral cleaning apparatus that is easy to carry and is stored hygienically.

It will be apparent to those skilled in the art which various modifications may be made to the above-described an embodiment of the present disclosure without departing from the spirit or scope of the invention. Thus, it is intended which the present invention cover all such modifications provided they come in the scope of the appended claims and their equivalents.

What is claimed is:

1. An oral cavity cleaning apparatus, comprising:
a body portion accommodating a driving portion therein;
a nozzle portion disposed at an end of the body portion and comprising a through hole; and
a cover member covering the through hole in the nozzle portion.

2. The oral cavity cleaning apparatus of claim 1, wherein the cover member comprises a cleaning member, and a sheet member impregnated with the cleaning member.

3. The oral cavity cleaning apparatus of claim 1, wherein the nozzle portion is detachably coupled to the body portion.

4. The oral cavity cleaning apparatus of claim 1, wherein the nozzle portion comprises a groove portion on a side thereof, and the cover member is inserted into the nozzle portion through the groove portion.

5. The oral cavity cleaning apparatus of claim 1, wherein the nozzle portion comprises a first leg portion and a second leg portion connected to the body portion, and
wherein the first leg portion and the second leg portion are disposed to face each other and are movable in a direction toward or away from each other.

6. The oral cavity cleaning apparatus of claim 5, wherein the cover member is disposed between the first leg portion and the second leg portion.

7. The oral cavity cleaning apparatus of claim 1, wherein the nozzle portion is rotatably coupled to the body portion.

8. An oral cavity cleaning apparatus, comprising:
a body portion accommodating a driving portion therein;
a cartridge portion disposed in a receiving space provided in the body portion and accommodating a cleaning member in liquid form; and
a discharge portion connected to the cartridge portion and discharging the cleaning member.

9. The oral cavity cleaning apparatus of claim 8, wherein the discharge portion comprises:
a discharge cover portion forming an outer shape of the discharge portion and allowing the cleaning member to move inward; and
a discharge body portion in communication with an inside of the discharge cover portion and comprising a discharge hole through which the cleaning member is sprayed outward.

10. The oral cavity cleaning apparatus of claim 8, further comprising a mounting portion provided by surrounding at least a portion of an outer surface by the body portion, and the discharge portion is disposed at the mounting portion.

11. The oral cavity cleaning apparatus of claim 10, wherein the discharge portion is slidably coupled to the body portion.

12. The oral cavity cleaning apparatus of claim 10, wherein the discharge portion is hinge-connected to the body portion.

13. The oral cavity cleaning apparatus of claim 8, wherein the body portion further comprises a body cover portion opening or closing the receiving portion.

14. The oral cavity cleaning apparatus of claim 8, further comprising an irradiation portion irradiating light of a predetermined wavelength.

15. The oral cavity cleaning apparatus of claim 14, wherein each of the discharge portion and the irradiation portion is disposed at one end of the body portion.
